(19) United States
(12) Patent Application Publication
(10) Pub. No.: US 2018/0021599 A1
(43) Pub. Date: Jan. 25, 2018

(54) APPARATUS FOR REMOVING FAT AND APPARATUS FOR SEPARATING CELL

(71) Applicant: MEDI FUTURES CO., LTD., Seoul (KR)

(72) Inventors: Ji-Hwan KIM, Busan (KR); Seong-Hyeong LEE, Changwon-si (KR)

(21) Appl. No.: 15/714,073

(22) Filed: Sep. 25, 2017

Related U.S. Application Data
(63) Continuation-in-part of application No. PCT/KR2016/002448, filed on Mar. 11, 2016.

(30) Foreign Application Priority Data
Mar. 26, 2015 (KR) 10-2015-0042595
Sep. 15, 2015 (KR) 10-2015-0130446

(51) Int. Cl. A61N 7/02; A61M 1/00

(52) U.S. Cl. CPC A61N 7/02; A61M 1/0001; A61N 2007/0008

(57) ABSTRACT

As various embodiments of the present disclosure related to an electronic fat removing apparatus using ultrasonic waves are described, a fat removing apparatus according to an embodiment may include: a fat removal body part; a handpiece including an ultrasonic wave generating unit electrically connected to the fat removal body part; a tip part provided in the handpiece; a temperature sensor unit for detecting the temperature of the tip part, the temperature sensor unit being provided in the tip part; an interlocked pump part for feeding cooling water to the tip part, the interlocked pump part being disposed in the fat removal body unit and being connected to the handpiece; and a controller for receiving a signal detected by the temperature sensor unit, and operating the interlocked pump part at a preconfigured temperature. In addition, there may be various other embodiments related to the fat removing apparatus.

### CROSS-REFERENCE TO RELATED APPLICATIONS

This Application is a continuation in-part application of PCT Application No. PCT/KR2016/002448 filed on Mar. 11, 2016, which claims the benefit of Korean Patent Application No. 10-2015-0042595 filed on Mar. 26, 2015, and Korean Patent Application No. 10-2015-0130446 filed on Sep. 15, 2015, the entire disclosures of which are incorporated herein by reference

### TECHNICAL FIELD

Various embodiments of the present disclosure relate to a fat removing apparatus for removing subcutaneous fat using ultrasonic waves, and a cell separating apparatus for separating a cell from fat cells.

### BACKGROUND ART

In general, examples of plastic surgeries for removing fat layers that have excessively been accumulated in a human body include liposuction and fat removal surgery. In particular, the fat removal surgery can easily remove, as needed, fat in a portion where it is difficult to remove fat thereof by exercise, diet therapy, and the like. That is, methods such as exercise and diet therapy reduce the sizes of fat cells, and thus, have difficulty in suppressing the fat layers from being thickened again later. However, the fat removal surgery reduces the number of fat cells, and thus, has an advantage in remarkably reducing the above problem.

The fat removal surgery is largely classified into a scheme based on a mechanical apparatus and a scheme based on an electronic apparatus, and the scheme based on a mechanical apparatus is classified into a dry plastic surgery and a wet plastic surgery. First, the scheme based on a mechanical apparatus, which was developed in the early 1980s, is a scheme of inserting a cannula having a rough surface into fat layers, and then moving the cannula forwardly and backwardly, thereby separating and destroying fat cells, and thus, removing fat.

However, this scheme has problems in that a patient feels large pain and suffers from a large amount of bleeding, and an operator is also required to use a lot of labor and has difficulty in accurately performing the surgery.

In order to solve the above problems, the wet plastic surgery has been developed.

The wet plastic surgery is a scheme of injecting a physiological saline solution containing local anesthetics, a vasoconstrictor, and the like, into fat layers to be removed, inflating the thicknesses of the fat layers up to several times the original thicknesses, and then removing fats in the same manner as that of the dry plastic surgery using the cannula.

Here, the cannula includes a tip.

As compared with the dry plastic surgery, the wet plastic surgery has many advantages in that the thicknesses of the fat layers to be operated are inflated up to several times the original thicknesses, thereby improving the accuracy of the surgery, and capillaries are contracted by the vasoconstrictor, thereby reducing the amount of bleeding generated during an operation. However, similar to the dry plastic surgery, the wet plastic surgery cannot solve problems of pain of a patient, a lot of labor of an operator, and the like because fat cells should be separated and destroyed by forwardly and backwardly moving the tip of the cannula having the rough surface.

Thus, in order to solve the above-mentioned problems, an electronic fat removing apparatus using ultrasonic waves has been developed. Such an ultrasonic wave fat removing apparatus uses an apparatus of converting ultrasonic electronic energy into mechanical energy corresponding to vibration. First, the tip of the cannula, which generates ultrasonic vibrations using a piezoelectric element vibrating when an alternating current is applied thereto, is inserted into the fat layers, and is then vibrated. Bubbles are generated in the fat layers by the ultrasonic vibrations of the tip, fat tissues are separated by cavitation, micromechanical destruction, a thermal effect, and the like, resulting from the generation of the bubbles, and the fat tissues are removed, so that fat is removed. At this time, the thermal effect serves to melt the fat.

### SUMMARY

However, the conventional ultrasonic fat removing apparatus has disadvantages in that, in the case of a cannula for melting fat using ultrasonic waves in order to remove subcutaneous fat, a lot of heat is generated at the tip of the cannula in order to obtain thermal effects, thereby causing the skin and the muscle of a user to be burned, and after the fat removal surgery, pain and swelling of a patient occurs due to burn inflammation.

Finally, in order to overcome such disadvantages, a cooling apparatus for cooling heat that is generated by the tip of the cannula is required.

Thus, various embodiments of the present disclosure provide a fat removing apparatus that has a tip part of a handpiece capable of cooling heat generated during a vibration caused by ultrasonic waves, so that a skin layer and a muscle layer of the patient can be prevented from being burned due to the heat generated by the tip, and at the same time, the pain, and the swelling resulting from the burn inflammation of a patient after the surgery can be prevented from occurring.

Further, in general, stem cell related separation methods for helping treatment by separating cells from fat cells or other tissues of a person are being rapidly developed. In particular, a method of melting a cell using the existing enzyme (collagenase), and extracting a by-product generated by the melting is used as a method of separating a stem cell from fat cells of a person. However, unlike acidic solutions or alkali solutions, the enzyme (collagenase) is not neutralized, and has toxicity of the enzyme itself. The enzyme, which is not neutralized, can melt other proteins and fats in a human body, and the conventional stem cell separating equipment has a disadvantage in that a large amount of time is consumed for washing the enzyme (collagenase) several times so as to remove the remaining enzymes.

That is, in a method of separating cells using the existing enzyme (collagenase), fat and cells collected from a human body are received in a container having a constant size, and an enzyme (collagenase) is added to the container, so that the cells are melted at a temperature of 38 degrees to 39 degrees, which is similar to that of a human body, for about 40 minutes. Thereafter, the cells are centrifuged so that the stem cells and other cells are separated therefrom.

Here, in order to neutralize the remaining enzyme (collagenase), the remaining enzyme is washed several times using normal saline, and the stem cells are then selected again.

In this way, the existing cell separating method includes melting decayed fat cells and cells by the enzyme (collagenase) for 40 minutes, and then neutralizing the enzyme (collagenase) in normal saline. At this time, the time required may be 20 minutes or one hour or longer. This is a time to be used due to the enzyme.

Thus, various embodiments of the present disclosure provide an ultrasonic cell separating apparatus which separates cells by vibrations caused by ultrasonic waves using a tip part of a handpiece instead of the enzyme (collagenase), thereby reducing the time consumed for melting the existing enzyme (collagenase) and the time consumed for neutralization.

According to various embodiments of the present disclosure, an electronic fat removing apparatus using ultrasonic waves may include: a fat removal body unit; a handpiece including an ultrasonic wave generating unit electrically connected to the fat removal body unit; a tip part provided in the handpiece; a temperature sensor unit that detects the temperature of the tip part, the temperature sensor unit being provided in the tip part; an interlocked pump unit that transfers cooling water to the tip part, the interlocked pump unit being connected to the handpiece; and a controller that receives a signal detected by the temperature sensor unit, and operates the interlocked pump unit at a preconfigured temperature.

According to various embodiments of the present disclosure, an electronic fat removing apparatus using ultrasonic waves may include: a fat removal body unit; a handpiece including an ultrasonic wave generating unit electrically connected to the fat removal body unit; a tip part that receives ultrasonic vibrations of the ultrasonic wave generating unit and vibrates, the tip part being provided in the handpiece; a temperature sensor unit that detects the temperature of the tip part, which rises according to ultrasonic waves, the temperature sensor unit being provided in the tip part; an interlocked pump unit that transfers cooling water to the tip part so as to cool the temperature of the tip part, the interlocked pump unit being disposed in the fat removal body unit and being connected to the handpiece; a cooling unit that cools the cooling water, the cooling unit being connected to the interlocked pump unit; and a controller that receives a signal detected by the temperature sensor unit, and operates the interlocked pump unit at a preconfigured temperature.

According to various embodiments of the present disclosure, a cell separating apparatus may include: a handpiece having an ultrasonic wave generating unit formed therein; a tip part disposed in the handpiece; and a storage bottle that receives fat cells and cells, wherein the storage bottle is coupled to the handpiece, and the tip part can be inserted into the storage bottle to melt and separate the fat cells and the cells by ultrasonic waves of the ultrasonic wave generating unit.

According to various embodiments of the present disclosure, heat generated during vibration can be cooled by cooling water by transferring ultrasonic vibrations of the ultrasonic wave generating unit to tip of the handpiece, so that the heat of the tip inserted into the fat layers of a patient is prevented from being generated. Accordingly, a skin layer and a muscle layer of the patient can be prevented from being burned, pain of the patient can be prevented from occurring, and swelling caused by burn inflammation can be prevented from occurring.

That is, subcutaneous fat can be quickly and efficiently reduced without burning the skin layer and the muscle layer of the patient.

Further, cells are separated from each other by ultrasonic waves without the enzyme (collagenase) so that a large amount of time required for melting or neutralizing the existing enzyme (collagenase) can be reduced to several minutes. Thus, the cells can be efficiently separated from the fat cells.

### BRIEF DESCRIPTION OF THE DRAWINGS

FIG. 1 illustrates a configuration of a fat removing apparatus according to various embodiments of the present disclosure;

FIG. 2 illustrates an operation state of the fat removing apparatus according to various embodiments of the present disclosure;

FIG. 3 is a side sectional view illustrating an operation state of the fat removing apparatus according to various embodiments of the present disclosure;

FIG. 4 is an enlarged side sectional view illustrating part A in FIG. 3;

FIG. 5 is a side sectional view illustrating a storage bottle among components of a cell separating apparatus according to various embodiments of the present disclosure;

FIG. 6 is a side sectional view illustrating a coupling state between the storage bottle and a tip part disposed in a handpiece among the components of the cell separating apparatus according to various embodiments of the present disclosure;

FIG. 7A is a view illustrating a state before the tip part of the handpiece is inserted into the storage bottle containing the fat cells and the cells, the volume of which is 25 cc, according to various embodiments of the present disclosure;

FIG. 7B is a view illustrating a process of inserting the tip part of the handpiece into the storage bottle containing the fat cells and the cells, the volume of which is 25 cc, and then melting the cells according to various embodiments of the present disclosure;

FIG. 8 is a view illustrating a state in which the melted cells are separated from the remaining cells using a centrifuge according to various embodiments of the present disclosure;

FIG. 9 is a view illustrating values obtained by measuring the separated cells by a cell counter according to various embodiments of the present disclosure;

FIG. 10 is a side sectional view illustrating a cell separating apparatus according to another embodiment of the present disclosure; and

FIG. 11 is a side sectional view illustrating a storage bottle and peripheral components according to another embodiment of the present disclosure.

### DETAILED DESCRIPTION

Terms used in various embodiments of the present disclosure will be briefly described, and various embodiments of the present disclosure will be described in detail.

With respect to the terms in the various embodiments of the present disclosure, the general terms which are currently and widely used are selected in consideration of functions of structural elements in the various embodiments of the present disclosure. However, meanings of the terms may be changed according to an inventor's intention, a judicial precedent, appearance of a new technology, and the like. Further, in a certain case, a term arbitrarily selected by the applicant may be used. In such a case, the meaning of the term will be described in detail at the corresponding part in the description of the present disclosure. Thus, the terms used in various embodiments of the present disclosure should be defined based on the meanings of the terms and the overall contents of the embodiments of the present disclosure instead of simple titles of the terms.

Although the terms including an ordinal number such as first, second, etc. can be used for describing various elements, the structural elements are not restricted by the terms. The terms are used merely for the purpose to distinguish an element from the other elements. For example, a first element could be termed a second element, and similarly, a second element could be also termed a first element without departing from the scope of the present disclosure.

A fat removing apparatus and a cell separating apparatus according to various embodiments of the present disclosure may be one of the above-mentioned apparatuses or a combination of one or more thereof. Further, it is obvious to those skilled in the art that the fat removing apparatus and the cell separating apparatus according to various embodiments of the present disclosure are not limited to an apparatus used for the fat removal surgery and the cell separation. For example, when used for medical care, the fat removing apparatus and the cell separating apparatus can be applied to various kinds of dental treatments, the osteotomy and the saucerization corresponding to orthopaedic surgeries.

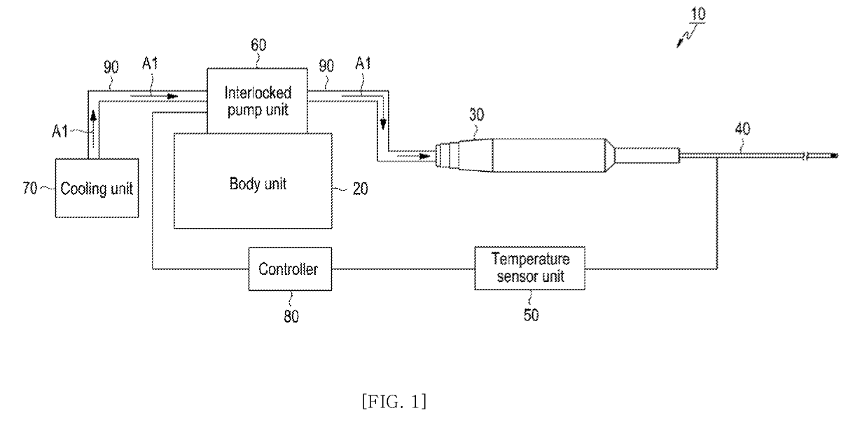

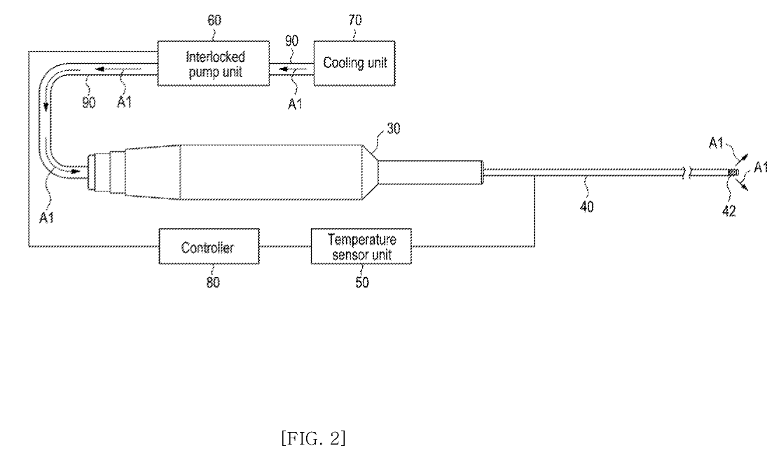

FIG. 1 illustrates a configuration of a fat removing apparatus 10 according to various embodiments of the present disclosure, and FIG. 2 illustrates an operation state of the fat removing apparatus 10 according to various embodiments of the present disclosure.

Referring to FIGS. 1 and 2, the configuration of the electronic fat removing apparatus 10 using ultrasonic waves will be described. The fat removing apparatus 10 includes: a fat removal body unit 20; a handpiece 30; a tip part 40; a temperature sensor unit 50, an interlocked pump unit 60; a cooling unit 70; and a controller 80.

The fat removal body unit 20 is configured to generate an ultrasonic electrical signal so as to simultaneously transfer the generated ultrasonic electrical signal to the handpiece 30 while being electrically connected to the handpiece 30 which will be described below.

The handpiece 30 is electrically connected to the fat removal body unit 20 so as to receive the ultrasonic electrical signal of the fat removal body unit 20, generate ultrasonic vibrations, and transfer the generated ultrasonic vibrations to the tip part 40 which will be described below.

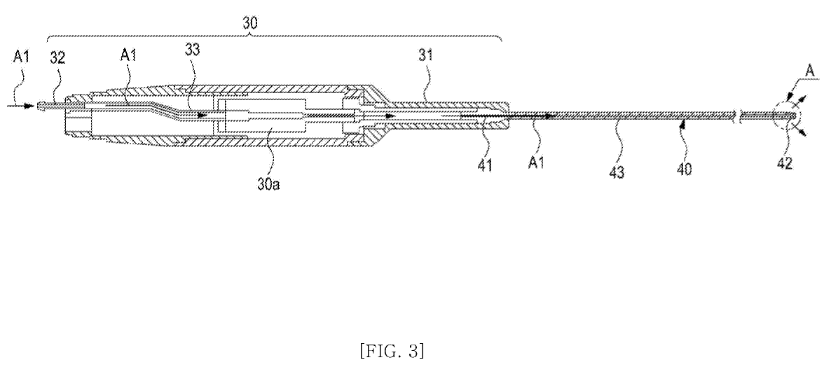

The handpiece 30 has an ultrasonic wave generating unit 30a (illustrated in FIG. 3) to generate ultrasonic vibrations. The handpiece 30 may be referred to as a base unit.

The ultrasonic wave generating unit 30a (illustrated in FIG. 3) includes a piezoelectric element to generate ultrasonic vibrations in a piezoelectric scheme.

In the present embodiment, although the piezoelectric element is exemplified as the ultrasonic wave generating unit, the present disclosure is not limited thereto. That is, a device for generating ultrasonic waves in different schemes other than the piezoelectric scheme may also be adopted as the ultrasonic wave generating unit 30a (illustrated in FIG. 3).

The tip part 40 is disposed in the handpiece 30 to receive the ultrasonic vibrations so as to vibrate. The tip part 40 may be referred to as a probe.

The temperature sensor unit 50 is disposed in the tip part 40 so as to detect the temperature of the tip part 40, which rises according to the ultrasonic vibrations.

While being connected to the handpiece 30 by a cooling hose 90, the interlocked pump unit 60 is disposed in the fat removal body unit 20 so as to transfer cooling water A1 of the cooling unit 70 to the tip part 40 so as to cool the tip part 40, the temperature of which rises.

While being connected to the interlocked pump unit 60 by the cooling house 90, the cooling unit 70 is configured to supply the cooling water A1 to the interlocked pump unit 60.

The controller 80 is configured to receive a signal detected by the temperature sensor unit 50 so as to operate the interlocked pump unit 60 at a preconfigured temperature.

The preconfigured temperature may be 80 degrees or higher. That is, the temperature sensor unit 50 detects the temperature of the tip part 40, and applies the detected signal to the controller 80, the controller 80 operates the interlocked pump unit 60 when the temperature of the tip part 40 is 80 degrees or higher, and the interlocked pump unit 60 supplies the cooling water A1 to the handpiece 30. The handpiece 30 can transfer the cooling water A1 to the tip part 40 so as to cool the temperature of the tip part 40, thereby preventing a skin layer and a muscle layer of a patient from being burned.

Further, a tip coupling part 31 coupled to the tip part 40 is disposed at one end of the handpiece 30, and a pump connection part 32 is disposed at the other end of the handpiece 30 so as to be connected to the interlocked pump unit 60.

That is, the tip coupling part 31 is coupled to a mounting part 41 formed in the tip part 40 so as to receive the cooling water A1 supplied to the handpiece 30. The pump connection part 32 is connected to the interlocked pump unit 60 so as to supply the cooling water A1 of the cooling unit 70 to the inside of the handpiece 30.

A cooling water transfer channel 33 is disposed in the handpiece 30 so as to transfer the cooling water A1 to the tip part 40 while connecting the tip coupling part 31 and the pump connection part 32 to each other.

Further, the mounting part 41 is disposed at one end of the tip part 40 such that the tip part 40 is mounted to the tip coupling part 31, and at least one discharge hole 42 is formed at the other end of the tip part 40 such that the cooling water A1 can be discharged to the outside through the discharge hole 42.

That is, the tip part 40 receives and moves the cooling water A1 supplied to the handpiece 30 while cooling the rising temperature, and then, discharges the cooling water A1 to the outside of the tip part 40 through the discharge hole 42.

Since a cooling water transfer channel 43 is disposed inside the tip part 40 such that the cooling water A1 introduced through the handpiece 30 can move therethrough, the cooling water A1 introduced into the tip part 40 moves through the cooling water transfer channel 43 while cooling the temperature of the tip part 40, and then is discharged to the outside of the tip part 40 through the discharge hole 42.

The cooling water A1 may be composed of saline solutions. In the present embodiment, although saline solutions are exemplified as the cooling water A1, the present disclosure is not limited thereto. That is, the cooling water A1 may be harmful or harmless to a human body.

In this state, a process of assembling the fat removing apparatus 10 will be described in more detail. First, as mentioned in FIGS. 1 and 2 above, the tip part 40 is coupled to the handpiece 30. The mounting part 41 of the tip part 40 is coupled to the tip coupling part 31 disposed at one end of the handpiece 30. The cooling hose 90 is connected to the pump connection part 32 disposed at the other end of the handpiece 30, and the interlocked pump unit 60 is connected to the cooling hose 90. The interlocked pump unit 60 is connected to the cooling unit 70 using yet another cooling hose 90.

At this time, the tip part 40 includes the temperature sensor unit 50 for detecting the temperature of the tip part 40, and the temperature sensor unit 50 is electrically connected to the controller 80. The fat removal body unit 20 is electrically connected to the handpiece 30 to apply ultrasonic signals of the fat removal body unit 20 to the handpiece 30.

In this state, a process of operating the fat removing apparatus 10 will be described below in detail. FIG. 3 is a side sectional view illustrating an operation state of the fat removing apparatus 10 according to various embodiments of the present disclosure, and FIG. 4 is an enlarged side sectional view of part A of FIG. 3.

First, as mentioned in FIGS. 1 and 2 above, the skin of a patient who wants to remove fat thereof is slightly cut, and the tip part 40 disposed in the handpiece 30 is then inserted into fat layers. A medicine (not illustrated) for facilitating removing of fat is injected through the tip part 40. The medicine is obtained by diluting anesthetics, a solution for preventing bleeding, and the like with normal saline.

That is, in the present embodiment, when the medicine is injected into fat of a patient, damage to blood vessels and tissues are reduced, and frictional resistance of the tip part 40 and skin resistance are reduced, so that the surgery can be performed using a small amount of labor.

In this case, current is supplied to the fat removal body unit 20, the fat removal body unit 20 generates ultrasonic electrical signals, and the ultrasonic electrical signals are adopted as the ultrasonic wave generating unit 30a (illustrated in FIG. 3) provided in the handpiece 30. The ultrasonic wave generating unit 30a (illustrated in FIG. 3) generates ultrasonic vibrations in the piezoelectric scheme, and the ultrasonic vibrations are transferred to the tip part 40 while the tip part 40 vibrates forwardly and backwardly. Due to the forward and backward ultrasonic vibrations of the tip part 40, fat cells of the fat layers are separated and destroyed by cavitation, micromechanical destruction, a thermal effect, and the like, resulting from the generation of bubbles, so that the fat is separated. Such separated fat is removed by an operation of a vacuum pump (not illustrated) disposed in the fat removal body unit 20, is introduced into the tip part 40, and at the same time, is collected in a fat storage container (not illustrated) disposed in the fat removal body unit 20.

At this time, when the tip part 40 is inserted into the skin of the patient, and then ultrasonically vibrates, the tip part 40 emits heat due to the forward and backward ultrasonic vibrations. The heat of the tip part 40 is detected by the temperature sensor unit 50 disposed in the tip part 40. When the tip part 40 generates excessive heat due to the ultrasonic vibrations, the temperature sensor unit 50 detects the excessive heat so as to apply the same to the controller 80, as illustrated in FIG. 3. The controller 80 operates the interlocked pump unit 60 when the temperature is equal to or higher than a preconfigured temperature. That is, the controller 80 operates the interlocked pump unit 60 when it is measured that the temperature of the tip part 40 is about 80 degrees or higher. The interlocked pump unit 60 moves and supplies the cooling water A1 of the cooling unit 70 to the handpiece 30, and supplies the cooling water A1 to the tip part 40 using the cooling water transfer channel 33 disposed inside the handpiece 30.

Since the cooling water transfer channel 43 is disposed inside the tip part 40 such that the cooling water A1 introduced through the cooling water transfer channel 33 of the handpiece 30 can move therethrough, the cooling water A1 moves through the cooling water transfer channel 43 of the tip part 40 simultaneously while cooling the rising temperature of the tip part 40.

Further, as illustrated in FIG. 4, the cooling water A1 is discharged to the outside of the tip part 40 through the discharge hole 42 of the tip part 40.

The tip part 40 cooled in this manner removes the cooling water A1 and the fat discharged from the skin of the patient, and the cooling water A1 and the fat are moved and collected in the storage container (not illustrated) or a syringe (not illustrated) of the fat removal body unit 20.

In this way, the conventional fat removing apparatus (not illustrated) using ultrasonic waves has a disadvantage in that when vibrating forwardly and backwardly by ultrasonic waves, a tip part (not illustrated) generates excessive heat, and thus, a skin layer and a muscle layer of a patient are burned.

Thus, in order to overcome the disadvantage, the present disclosure includes: the temperature sensor unit 50 for detecting the temperature generated during the ultrasonic vibrations of the tip part 40; and the controller 80 for controlling the same, wherein the controller 80 is configured to supply the cooling water A1 for cooling the rising temperature of the tip part 40. Therefore, the tip part 40 is cooled by the cooling water A1, thereby preventing burning of the skin layer and the muscle layer of the patient, and accordingly, preventing the occurrence of swelling and pain of a patient resulting from burn inflammation as well as preventing the occurrence of pain after the fat removal surgery.

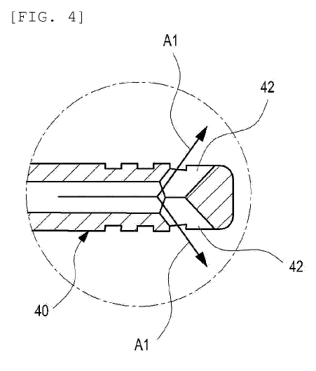

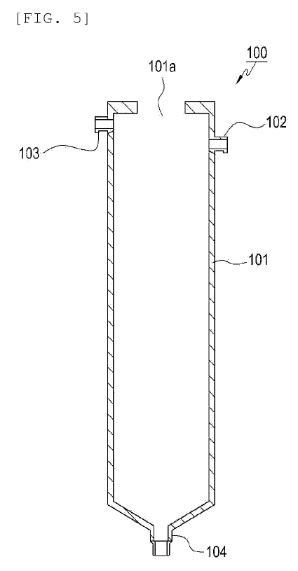

FIG. 5 is a side sectional view illustrating the storage bottle 100 of the cell separating apparatus according to various embodiments of the present disclosure, and FIG. 6 is a side sectional view illustrating a coupling state between the storage bottle 100 and the tip part disposed in the handpiece among components of the cell separating apparatus according to various embodiments of the present disclosure.

Referring to FIGS. 5 and 6, the configuration of the electronic cell separating apparatus 110 using ultrasonic waves will be described. The cell separating apparatus 110 includes: a handpiece 30; a tip part 40; a storage bottle 100; a temperature sensor unit 50, an interlocked pump unit 60; a cooling unit 70; and a controller 80.

The handpiece 30 is electrically connected to a body unit (not illustrated) of the cell separating apparatus 110 so as to receive the ultrasonic electrical signal generated by the body unit (not illustrated) of the cell separating apparatus 110, generate ultrasonic vibrations, and transfer the generated ultrasonic vibrations to the tip part 40 which will be described below.

The handpiece 30 has an ultrasonic wave generating unit 30a (illustrated in FIG. 3) to generate ultrasonic vibrations. The handpiece 30 may be referred to as a base unit.

The ultrasonic wave generating unit 30a (illustrated in FIG. 3) includes a piezoelectric element to generate ultrasonic vibrations in the piezoelectric scheme. In a conventional ultrasonic apparatus, vibrations caused by ultrasonic waves are generated not only at one end of a tip part in contact with fat cells but also in the middle of the tip part 40 due to a wavelength of the ultrasonic waves. However, in the ultrasonic wave generating unit 30a according to an embodiment of the present disclosure, vibrations caused by ultrasonic waves can be intensively generated only at one end of the tip part 40 (i.e., a portion where an auxiliary member 50 is disposed). If the ultrasonic wave generating unit 30a is configured such that a distance from the ultrasonic wave generating unit 30a to the one end of the tip part 40 corresponds to a wavelength or a half wavelength of ultrasonic waves by controlling a vibrator included in the ultrasonic wave generating unit 30a, vibrations caused by ultrasonic waves can be generated only at the one end of the tip part 40. When fat cells are separated, only the one end of the tip part 40 is not immersed in the fat cells but a predetermined length of the tip part 40 is immersed in the fat cells. In this case, heat is generated in a middle portion of the tip part 40, fat cells in contact with the portion may be damaged. Therefore, the above-described feature according to an embodiment of the present disclosure can be helpful in separating fat cells with cell walls alive without damaging the fat cells.

In the present embodiment, although the piezoelectric element is exemplified as the ultrasonic wave generating unit 30a, the present disclosure is not limited thereto. That is, a device for generating ultrasonic waves in different schemes other than the piezoelectric scheme may be also adopted as the ultrasonic wave generating unit 30a (illustrated in FIG. 3).

The tip part 40 is disposed in the handpiece 30 to receive the ultrasonic vibrations so as to vibrate. The tip part 40 may be referred to as a probe.

The temperature sensor unit 50 (illustrated in FIG. 2) is disposed in the tip part 40 so as to detect the temperature of the tip part 40, which rises according to the ultrasonic vibrations.

While being connected to the handpiece 30 by a cooling hose 90, the interlocked pump unit 60 (illustrated in FIG. 2) is disposed in a body unit (not illustrated) of the cell separating apparatus 110 so as to transfer cooling water A1 of the cooling unit 70 (illustrated in FIG. 2) to the tip part 40 so as to cool the tip part 40, the temperature of which rises.

While being connected to the interlocked pump unit 60 by the cooling house 90, the cooling unit 70 (illustrated in FIG. 2) is configured to supply the cooling water A1 to the interlocked pump unit 60 (illustrated in FIG. 2).

The controller 80 (illustrated in FIG. 2) is configured to receive a signal detected by the temperature sensor unit 50 so as to operate the interlocked pump unit 60 at a preconfigured temperature.

The preconfigured temperature may be 80 degrees or higher. That is, the temperature sensor unit 50 (illustrated in FIG. 2) detects the temperature of the tip part 40, and applies the detected signal to the controller 80, the controller 80 (illustrated in FIG. 2) operates the interlocked pump unit 60 (illustrated in FIG. 2) when the temperature of the tip part 40 is 80 degrees or higher, and the interlocked pump unit 60 supplies the cooling water A1 to the handpiece 30. The handpiece 30 can transfer the cooling water A1 to the tip part 40 so that the cooling water A1 cools the temperature of the tip part 40.

Further, a tip coupling part 31 coupled to the tip part 40 is disposed at one end of the handpiece 30, and a pump connection unit 32 is disposed at the other end of the handpiece 30 to be connected to the interlocked pump unit 60 (illustrated in FIG. 2).

That is, the tip coupling part 31 is coupled to a mounting part 41 formed in the tip part 40 so as to receive the cooling water A1 supplied to the handpiece 30. The pump connection part 32 is connected to the interlocked pump unit 60 (illustrated in FIG. 2) such that the cooling water A1 of the cooling unit 70 (illustrated in FIG. 2) is supplied therethrough to the inside of the handpiece 30.

A cooling water transfer channel 33 is disposed inside the handpiece 30 such that the cooling water A1 can be transferred to the tip part 40 therethrough simultaneously while the tip coupling part 31 and the pump connection part 32 are connected to each other through the cooling water transfer channel 33.

Further, the mounting part 41 is disposed at one end of the tip part 40 such that the tip part 40 is mounted to the tip coupling part 31, and at least one discharge hole 42 is formed at the other end of the tip part 40 such that the cooling water A1 can be discharged to the outside therethrough.

That is, the tip part 40 receives and moves the cooling water A1 supplied to the handpiece 30 simultaneously while the cooling water A1 cools the rising temperature, and then discharges the cooling water A1 to the outside of the tip part 40 through the discharge hole 42.

Since a cooling water transfer channel 43 is disposed inside the tip part 40 such that the cooling water A1 introduced through the handpiece 30 can move therethrough, the cooling water A1 introduced into the tip part 40 moves through the cooling water transfer channel 43 while cooling the temperature of the tip part 40, and is then discharged to the outside of the tip part 40 through the discharge hole 42.

The cooling water A1 may be made of saline solutions. In the present embodiment, although saline solutions are exemplified as the cooling water A1, the present disclosure is not limited thereto. That is, the cooling water A1 may be harmful or harmless to a human body.

Meanwhile, the components, such as the interlocked pump unit 60, the discharge hole 42, the temperature sensor unit 50, and the cooling unit 70, for supplying the cooling water A1 into the tip part 40 may not be prerequisites for the cell separating apparatus.

Further, the storage bottle 100 can contain fat cells. In this state, while the storage bottle 100 is coupled to the handpiece, the tip part is inserted into the storage bottle 100, so that the fat cells in the storage bottle 100 are separated by the ultrasonic vibrations of the ultrasonic wave generating unit.

As mentioned in FIG. 5 above, the storage bottle 100 may include a cylindrical body unit 101, an injection hole 102, an air channel 103, and a discharge hole 104.

The cylindrical body unit 101 may contain the fat cells.

The injection hole 102 may be disposed on the side surface of the cylindrical body unit 101 such that a predetermined amount (e.g., 25 cc) of the fat cells is injected into the cylindrical body unit 101, and the cylindrical body unit 101 thus contains the fat cells.

The air channel 103 is an air inlet to introduce air and may be disposed on the side surface of the cylindrical body unit 101 so as to facilitate air circulation in the cylindrical body unit 101. Otherwise, the air channel 103 may function to inject a drug or water for facilitating the separation of fat cells. For example, a material in the storage bottle 100 may become dry and hard during a process of separating fat cells. In this case, since the discharge hole 104 and the injection hole 102 are connected to each other through a tube 108, this problem can be solved by supplying water through the air channel 103.

The discharge hole 104 is disposed below the cylindrical body unit 101 such that a separated material or a material in the storage bottle 100 can be discharged to the outside therethrough. The discharge hole 104 and the injection hole 102 may be connected to each other through the tube 108. In this case, materials in the cylindrical body unit 101 can be continuously circulated through the discharge hole 104 and the injection hole 102. Referring to FIG. 11, the tube 108 may be connected to a circulating pump 107. A material discharged through the discharge hole 104 can be transferred by the circulating pump 107 to the injection hole 102 positioned higher than the discharge hole 104. If a material in the storage bottle 100 is not circulated, fat cells are separated only at or around a portion of the tip part 40 where vibrations caused by ultrasonic waves are generated but may not be separated well in the other area. However, since the material in the storage bottle 100 is circulated by the tube 108 and the circulating pump 107, the fat cells can be uniformly separated.

Ultrasonic waves of the ultrasonic wave generating unit may have a wavelength of 22-29 KHz. The ultrasonic wave generating unit, which generates ultrasonic waves having a wavelength of 22-29 KHz, can separate human body-derived tissues from fat tissues in units of cells without damaging all cells except for fat cells as well as can separate the human body-derived tissues by modifying the amplitude of ultrasonic waves while being switched to one of stages 1-10 of a buffer (boost). In other words, Stromal Vascular Fractions (SVFs) can be separated from the fat tissues without using the existing enzyme (collagenase).

The present disclosure can separate human body-derived tissues in units of cells using ultrasonic waves of ultrasonic wave generation, separate cells from fat tissues, separates cell except for fat cells from fat tissues, separate SVFs from fat tissues, and separate regenerated cells, stem cells, or immature precursor cells from fat tissues.

In more detail, the fat cells of the fat tissues have a structure in which SVFs, fat, fibrosis (a protein material such as collagen) and water are organizationally combined. The SVFs are composed of various cells including fat stem cells. In a conventional technology, a coupling tissue including the SVFs and collagen is separated in units of cells through reacting and melting the collagen, which supports the cells to be attached, using the existing enzyme (collagenase) at a temperature of 36.5 degrees similar to the body temperature for 30 minutes. Thus, the fat stem cells can be acquired. Accordingly, the present disclosure can effectively acquire the SVFs from the fat tissues for only several minutes through destroying and decomposing the fat cells and the collagen through ultrasonic waves in units of cells, and, thus, the fat stem cells can be acquired.

In this state, a process of assembling the cell separating apparatus 110 will be described in more detail. First, as mentioned in FIG. 6 above, the tip part 40 is coupled to the handpiece 30. The mounting part 41 of the tip part 40 is coupled to the tip coupling part 31 disposed at one end of the handpiece 30. The cooling hose 90 is connected to the pump connection part 32 disposed at the other end of the handpiece 30, and the interlocked pump unit 60 is connected to the cooling hose 90. The interlocked pump unit 60 is connected to the cooling unit 70 using yet another cooling hose 90.

At this time, the tip part 40 includes the temperature sensor unit 50 for detecting the temperature of the tip part 40, and the temperature sensor unit 50 is electrically connected to the controller 80. A body unit (not illustrated) of the cell separating apparatus is electrically connected to the handpiece 30 so as to apply ultrasonic signals of the body unit to the handpiece 30.

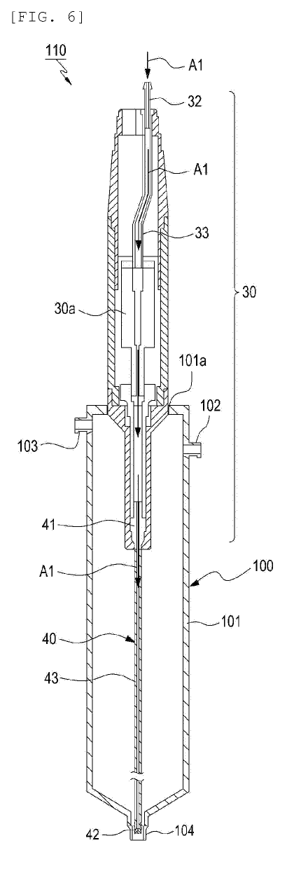

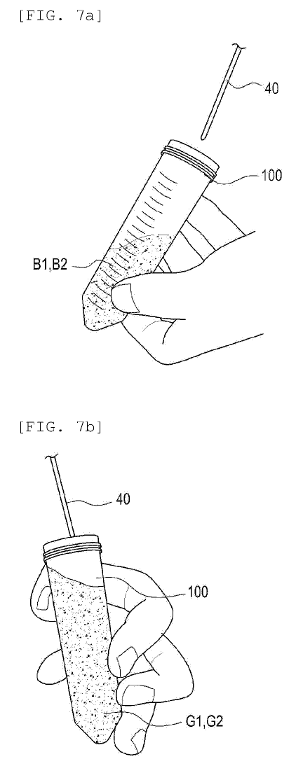

FIG. 7A illustrates a state before the tip part 40 of the handpiece 30 is inserted into the storage bottle 100 containing fat cells and cells, the volume of which is 25 cc, according to various embodiments of the present disclosure, and FIG. 7B illustrates a state in which the tip part of the handpiece is inserted into the storage bottle 100 containing fat cells and cells, the volume of which is 25 cc, and the cells are then melted. FIG. 8 is a view illustrating a state in which the melted cells are separated from the remaining cells using a centrifuge according to various embodiments of the present disclosure.

As illustrated in FIG. 7A, the fat cells, the volume of which is 25 cc, are injected into the injection hole 102 formed on the side surface of the cylindrical body unit 101 of the storage bottle 100.

In this case, current is supplied to the body unit (not illustrated) of the cell separating apparatus 110, the body unit generates ultrasonic electrical signals, and the ultrasonic electrical signals are applied to the ultrasonic wave generating unit 30a (illustrated in FIG. 6) disposed in the handpiece 30. The ultrasonic wave generating unit 30a (illustrated in FIG. 6) generates ultrasonic vibrations in the piezoelectric scheme, and the ultrasonic vibrations are transferred to the tip part 40 while the tip part 40 vibrates forwardly and backwardly, as illustrated in FIG. 7B. The fat cells can be decomposed due to the vibrations caused by the forward and backward ultrasonic waves of the tip part 40. In this case, the ultrasonic waves are transferred to one end of the tip part 40, and vibrations are generated by the ultrasonic waves through the auxiliary member 50, and then, a cavitation process is performed, and, thus, it is possible to separate fat stem cells G2 from the fat cells just by generating ultrasonic waves. Details of the auxiliary member 50 will be described later.

That is, the fat cells and collagen in the storage bottle 100 are destroyed and decomposed in units of cells through ultrasonic waves. In other words, the storage bottle 100 can decompose the fat cells in the storage bottle 100 into fat stem cells through ultrasonic waves only for several minutes (e.g., about 5 minutes). In this case, a filter 102a provided at the injection hole 102 may separate fibrosis from the other materials. The fibrosis functions to organizationally connect fat and SVFs like a branch. Thus, it is a material unnecessary for the extraction of fat stem cells. Thereafter, the materials remaining in the cylindrical body unit 101 may be separately collected and transferred into a tube for stem cell. Then, the materials may be separated to fat stem cells G2 and other cells G1 for about 2 minutes using a centrifuge as illustrated in FIG. 8. Through centrifugation, the fat stem cells G2 are disposed at the lowermost portion and the other cells G1 may be composed of water, broken fat, oil, etc.

Further, if the separated fat stem cells are extracted using a syringe, the separation of the fat stem cells can be completed within about 10 minutes or less. In this way, the extracted cells are put into a separation kit (not illustrated), and the number of extracted cells is then measured. That is, the number of the separated cells is measured by the cell counter (not illustrated).

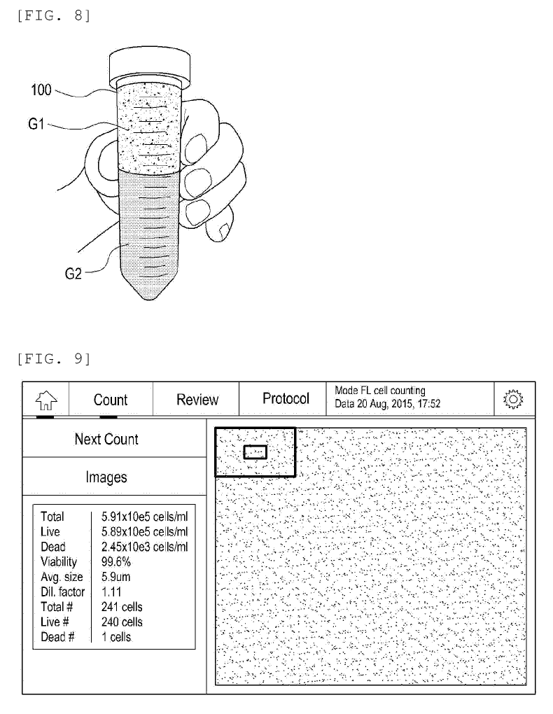

As illustrated in FIG. 9, in connection with the value measured by the cell counter, since 589000 cells among the total of 590000 cells are live, the live cell ratio is measured to be 99.6%. That is, it is measured that the cells are alive at a probability of 99.6%.

In other words, the coupling tissue between the cells and the collagen is separated in units of cells through reacting and melting the collagen, which supports the cells to be attached, using the existing enzyme (collagenase) at a temperature of 36.5 degrees similar to the body temperature for 30 minutes. However, the present disclosure can effectively acquire the SVFs from the fat tissues for only several minutes through destroying and decomposing the fat cells and the collagen through ultrasonic waves in units of cells.

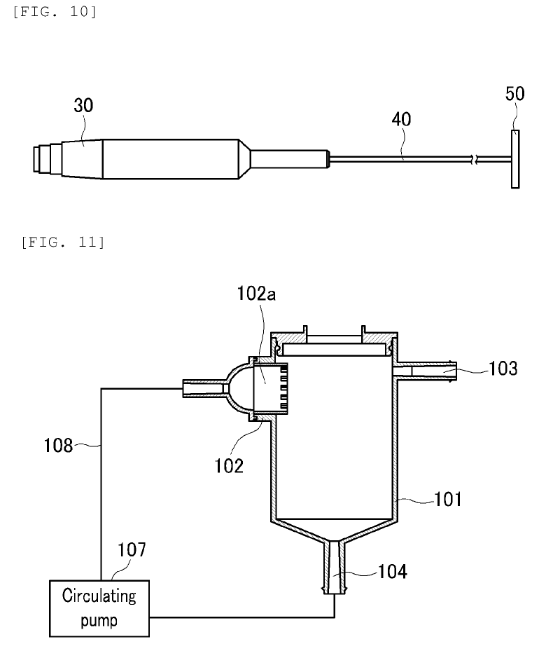

A cell separating apparatus according to another embodiment of the present disclosure may include the auxiliary member 50 at one end of the tip part 40 included in the handpiece 30. The auxiliary member 50 may have a smaller length than the tip part 40. When the auxiliary member 50 is mounted on the one end of the tip part 40, it may be mounted to face a direction different from a longitudinal direction of the tip part 40. For example, the auxiliary member 50 may be mounted to be perpendicular to the longitudinal direction of the tip part 40 as illustrated in FIG. 10, but is not necessarily limited thereto.

The auxiliary member 50 may be formed of a solid material, and an edge of the auxiliary member 50 may be mounted on the tip part 40 to be to be separated at a predetermined distance from the one end of the tip part 40. For example, the auxiliary member 50 may be formed to have a panel shape as illustrated in FIG. 10, and a central point of the panel shape and the one end of the tip part 40 may be combined with each other. Herein, edges of the auxiliary member 50 may be separated at the same distance from the one end of the tip part 40. Otherwise, the auxiliary member 50 may be formed as multiple rods intersecting at a central point, or the intersection of the rods may be combined with the one end of the tip part 40. Besides, the auxiliary member 50 may be formed to have various shapes and sizes with the edges separated from the one end of the tip part 40. That is, if the auxiliary member 50 is mounted, points where cells of fat tissues are affected by ultrasonic waves of the handpiece 30 correspond to the edges of the auxiliary member 50. The auxiliary member 50 may function to enable fat tissues in the storage bottle 100 to receive ultrasonic vibrations at a position slightly separated from the one end of the tip part 40 of the handpiece 30 without directly receiving ultrasonic vibrations from the one end of the tip part 40.

The auxiliary member 50 functions as an artificial variable resistance to an ultrasonic frequency. Specifically, if the handpiece 30 is designed to generate a frequency of x kHz from the one end of the tip part 40, solid molecules constituting the auxiliary member 50 may function as a medium different from the existing medium for ultrasonic waves generated from the ultrasonic wave generating unit and being propagated to the one end of the tip part 40. Since the different mediums adjoin each other, at least one of absorption, scattering, and diffusion is carried out, and, thus, the auxiliary member 50 can function as a resistance to an ultrasonic frequency. Therefore, at a portion where the auxiliary member 50 and the tip part 40 are combined, a frequency of x kHz is generated. However, at an edge of the auxiliary member 50, an attenuated frequency of x-a kHz may be generated.

In order to separate fat stem cells with cell walls unbroken, it is necessary to generate ultrasonic waves with an appropriate frequency. Ultrasonic waves have frequencies higher than the audible frequency range (20 Hz to 20 kHz) of human hearing, and, thus, the lowest frequency of the ultrasonic waves is 20 kHz. Therefore, a conventional ultrasonic wave generator can generate only ultrasonic waves with frequencies in a predetermined range, and due to the properties of ceramic constituting the ultrasonic wave generator, the ultrasonic wave generator can generate a frequency in the range of 25 to 30 kHz as the lowest frequency. However, conventional ultrasonic waves are so strong that not only cell walls but also cell nuclei of stem cells are broken. Therefore, a conventional medical ultrasonic wave generator has been used not for cell separation but for fat cell removal. In the case where a device in the form of the handpiece 30 is injected into a human body together with a drug to generate ultrasonic waves, fat cells can be divided. When the fat cells are divided, whether cell walls of the fat cells are dead or alive is not important because the divided fat cells are to be suctioned to the outside of the body. Therefore, in most of conventional methods for separating stem cells, fat stem cells are separated by injecting an enzyme such as collagenase. Since a frequency is too high to separate stem cells only with ultrasonic waves, the stem cells cannot survive. Otherwise, a conventional ultrasonic device uses ultrasonic waves for mixing fat cells and an enzyme such as collagenase finally needs to be used to acquire fat stem cells.

However, in the present disclosure, the auxiliary member 50 indirectly attenuates an ultrasonic frequency in an area where fat cells are in direct contact with ultrasonic waves, and, thus, it is possible to set the optimum ultrasonic frequency for the separation of fat stem cells with cell walls alive. Specifically, it is possible to generate sonic waves with somewhat lower frequencies than the lowest frequency of ultrasonic waves, and, thus, it is possible to separate fat stem cells with cell wall alive just by cavitation caused by the sonic waves. For example, at an edge of the auxiliary member 50 in contact with fat cells, sonic waves in the audible frequency band may be generated. The result shown in FIG. 9 was obtained by performing a stem cell separation experiment using the handpiece 30 equipped with the auxiliary member 50. Referring to FIG. 9, 589,000 cells among a total of 590,000 cells are live cells, and, thus, 99.6% of the cells were observed as being alive.

Therefore, according to the present disclosure, it is possible to separate stem cells with cell walls unbroken and alive from fat tissues just by generating ultrasonic waves without injecting an enzyme or drug for promoting the decomposition of the fat tissues.

The above-described fat removing apparatus and cell separating apparatus according to various embodiments of the present disclosure are not delimited by the above-described embodiments and drawings, and it is obvious to a person skilled in the art to which the present disclosure pertains that the fat removing apparatus and the cell separating apparatus can be variously substituted, modified and changed without departing from the technical scope of the present disclosure.

We claim:

1. A cell separating apparatus comprising:
a base unit having an ultrasonic wave generating unit formed therein;
a probe provided in the base unit to receive ultrasonic waves generated by the ultrasonic wave generating unit so as to vibrate; and
an auxiliary member mounted on one end of the probe where vibrations caused by the ultrasonic waves are generated and having a predetermined size and a predetermined length,
wherein since ultrasonic waves are generated by the ultrasonic wave generating unit after the auxiliary member is immersed in fat cells in a storage bottle, vibrations caused by the ultrasonic waves are transferred to the fat cells through the auxiliary member to decompose the fat cells into fat stem cells on the basis of the ultrasonic waves.

2. The cell separating apparatus of claim 1, wherein the auxiliary member is formed to have a panel shape.

3. The cell separating apparatus of claim 1, wherein the auxiliary member is formed to transfer vibrations to the fat cells at a position separated from the one end of the probe where the vibrations caused by the ultrasonic waves are generated.

4. The cell separating apparatus of claim 1, wherein the auxiliary member is mounted on the one end of the probe through a central point of the auxiliary member and an edge of the auxiliary member is separated from the one end of the probe.

5. The cell separating apparatus of claim 4, wherein edges of the auxiliary member are separated at the same distance from the one end of the probe.

6. The cell separating apparatus of claim 4, wherein a frequency caused by vibrations generated at the edge of the auxiliary member is lower than a frequency of ultrasonic waves generated by the ultrasonic wave generating unit.

7. The cell separating apparatus of claim 6, wherein the frequency caused by the vibrations generated at the edge of the auxiliary member is in the human audible frequency range.

8. The cell separating apparatus of claim 1, wherein the cell separating apparatus acquires fat stem cells from the fat cells just by generating ultrasonic waves without injecting an enzyme for promoting the decomposition of fat tissues into the storage bottle.

9. The cell separating apparatus of claim 1, further comprising:
a storage bottle which is coupled to the base unit such that the probe and the auxiliary member are disposed therein and contains fat cells therein.

10. The cell separating apparatus of claim 9, wherein the storage bottle comprises:
a cylindrical body unit including an opening formed at an upper end thereof to be coupled to the base unit;
an injection hole through which the fat cells are injected into the cylindrical body unit, the injection hole being disposed on the side surface of the cylindrical body unit; and
a discharge hole through which separated materials are discharged, the discharge hole being disposed at a lower portion of the cylindrical body unit.

11. The cell separating apparatus of claim 10, wherein the injection hole and the discharge hole are connected to each other through a tube to circulate a material in the storage bottle.

12. The cell separating apparatus of claim 11, further comprising:
a circulating pump configured to perform pumping to transfer a material discharged through the discharge hole to the injection hole through the tube.

13. The cell separating apparatus of claim 10, wherein the storage bottle further comprises:
a filter disposed at the injection hole and configured to filter fibrosis constituting the fat cells.

14. The cell separating apparatus of claim 10, wherein the storage bottle further comprises:
an air inlet provided to make it possible to selectively inject another material if necessary during a cell separation process of a material in the storage bottle.
